(19) United States
(12) Patent Application Publication
(10) Pub. No.: US 2021/0121222 A1
(43) Pub. Date: Apr. 29, 2021

(54) TISSUE ABLATION SYSTEM

(71) Applicant: INNOBLATIVE DESIGNS, INC., Chicago, IL (US)

(72) Inventor: Robert F. Rioux, Ashland, MA (US)

(21) Appl. No.: 16/633,765

(22) PCT Filed: Jul. 25, 2018
(86) PCT No.: PCT/US2018/043654

§ 371 Date: Jan. 24, 2020

Related U.S. Application Data
(60) Provisional application No. 62/537,413, filed on Jul. 26, 2017.

(51) Int. Cl. A61B 18/14; A61B 18/12

(52) U.S. Cl. CPC A61B 18/14; A61B 2018/0022; A61B 18/1206

(57) ABSTRACT

The present disclosure relates to a tissue ablation system including an ablation device having an expandable applicator tip configured to emit radio frequency (RF) energy for ablation and destruction of a target tissue.

### CROSS-REFERENCE TO RELATED APPLICATIONS

This application claims the benefit of, and priority to, U.S. Provisional Application No. 62/537,413, filed Jul. 26, 2017, the content of which is hereby incorporated by reference herein in its entirety.

### FIELD

The present disclosure relates generally to medical devices, and, more particularly, to a tissue ablation system including an ablation device having an expandable applicator tip configured to emit radio frequency (RF) energy for ablation and destruction of a target tissue.

### BACKGROUND

Cancer is a group of diseases involving abnormal cell growth with the potential to invade or spread to other parts of the body. Cancer generally manifests into abnormal growths of tissue in the form of a tumor that may be localized to a particular area of a patient's body (e.g., associated with a specific body part or organ) or may be spread throughout. Tumors, both benign and malignant, are commonly treated and removed via surgical intervention, as surgery often offers the greatest chance for complete removal and cure, especially if the cancer has not spread to other parts of the body. Electrosurgical methods, for example, can be used to destroy these abnormal tissue growths. However, in some instances, surgery alone is insufficient to adequately remove all cancerous tissue from a local environment.

For example, treatment of early stage breast cancer typically involves a combination of surgery and adjuvant irradiation. Unlike a mastectomy, a lumpectomy removes only the tumor and a small rim (area) of the normal tissue around it. Radiation therapy is given after lumpectomy in an attempt to eradicate cancer cells that may remain in the local environment around the removed tumor, so as to lower the chances of the cancer returning. However, radiation therapy as a post-operative treatment suffers various shortcomings. For example, radiation techniques can be costly and time consuming, and typically involve multiple treatments over weeks and sometimes months. Furthermore, radiation often results in unintended damage to the tissue outside the target zone. Thus, rather than affecting the likely residual tissue, typically near the original tumor location, radiation techniques often adversely affect healthy tissue, such as short and long-term complications affecting the skin, lungs, and heart.

Accordingly, such risks, when combined with the burden of weeks of daily radiation, may drive some patients to choose mastectomy instead of lumpectomy. Furthermore, some women (e.g., up to thirty percent (30%)) who undergo lumpectomy stop therapy before completing the full treatment due to the drawbacks of radiation treatment. This may be especially true in rural areas, or other areas in which patients may have limited access to radiation facilities.

### SUMMARY

The tissue ablation system of the present disclosure can be used during an ablation procedure to destroy the thin rim of marginal tissue around the cavity in a targeted manner. In particular, the present disclosure is generally directed to a cavitary tissue ablation system including an ablation device to be delivered into a tissue cavity and configured to emit non-ionizing radiation, such as radiofrequency (RF) energy, in a desired shape or pattern so as to deliver treatment for the ablation and destruction of a targeted portion of marginal tissue around the tissue cavity. The ablation device includes an expandable applicator tip configured to emit the RF energy in a desired pattern. The applicator tip includes a non-conductive flexible material configured to transition between a collapsed configuration, in which the tip can be delivered to a target site (i.e., a cavity or pocket), and an expanded configuration, in which the tip surface can better conform to the contour of the target tissue to be ablated, thereby allowing for improved contact and ablation/coagulation performance of the ablation device.

The system of the present invention is configured to provide a user with custom ablation shaping, which includes the creation of custom, user-defined ablation geometries depending on the target site. In particular, rather than simply providing a universal RF ablation shape or profile, the system allows for a user to customize the emission of energy to a targeted portion of marginal tissue within the cavity, which is particularly useful in instances in which non-uniform ablation is desired. The customized emission of energy may include a specific shape or geometry of emission, as well as time and depth of penetration of RF energy.

The devices, systems, and methods of the present disclosure can help to ensure that all microscopic disease in the local environment has been treated. This is especially true in the treatment of tumors that have a tendency to recur. Furthermore, by providing custom ablating shaping, in which the single ablation device may provide numerous RF energy emission shapes or profiles, the system of the present invention allows for non-uniform ablation to occur. This is particularly useful in controlling ablation shape so as to avoid vital organs and any critical internal/external structures (e.g., bone, muscle, skin) in close proximity to the tumor site, while ensuring that residual marginal tissue within the local environment has been treated.

The tissue ablation device of the present invention is generally in the form of a probe including an elongated shaft configured as a handle and adapted for manual manipulation and a flexible nonconductive distal portion or tip coupled to the shaft. The nonconductive distal tip is formed from a material having a low durometer and is deformable, thereby allowing for the distal tip to transition between a collapsed configuration, in which the distal tip has a first diameter, and an expanded configuration, in which the distal tip has a second diameter greater than the first diameter. The distal tip generally includes an interior chamber in which an inflatable inner balloon is positioned. The configuration of the distal tip is generally dependent on the current state of the inner balloon. In other words, the distal tip transitions to the expanded configuration in response to inflation of the inner balloon. Similarly, the distal tip transitions to the collapsed configuration in response to deflation of the inner balloon.

The distal tip includes an electrode array positioned along an external surface thereof. The distal tip, including the electrode array, can be delivered to and maneuvered within a tissue cavity (e.g., formed from tumor removal) when the distal tip is in the collapsed configuration and, upon transition to the expanded configuration, the distal tip is configured to ablate marginal tissue (via RF energy) immediately surrounding the tissue cavity in order to minimize recurrence of the tumor. The ablation device of the present invention is further configured to provide a user with custom ablation shaping, which includes the creation of custom, user-defined ablation geometries or profiles.

In one aspect, the electrode array includes a plurality of conductive wires electrically isolated and independent from one another. This design allows for each conductive wire to receive energy in the form of electrical current from a source (e.g., RF generator) and emit RF energy in response. The system may include a device controller, for example, configured to selectively control the supply of electrical current to each of the conductive wires. By allowing for independent control of each wire, the ablation system provides for custom ablation shaping to occur. In particular, the device controller allows for individual conductive wires, or a designated combination of conductive wires, to be controlled so as to result in the activation (e.g., emission of RF energy) of corresponding portions of the electrode array.

The device controller can selectively activate one or more of the electrode array portions (e.g., control the supply of electrical current to specific sets of conductive wires) so as to provide targeted delivery of RF energy from the ablation device in a desired pattern or shape. In addition to customizing the shape or geometry of RF energy emission from the ablation device, the device controller may be further configured to control particular ablation parameters, such as control of timing of the emission (e.g., length of time, intervals, etc.) as well as the depth of RF energy penetration.

In some embodiments, the ablation device is configured to provide RF ablation via a virtual electrode arrangement, which includes distribution of a fluid along an exterior surface of the distal tip and, upon activation of the electrode array, the fluid may carry, or otherwise promote, energy emitted from the electrode array to the surrounding tissue. For example, the nonconductive distal tip of the ablation device includes the interior chamber retaining at least the inner balloon, which may essentially act as a spacing member, and a hydrophilic insert surrounding a inner balloon. The interior chamber of the distal tip is configured to receive and retain a fluid (e.g., saline) therein from a fluid source. The hydrophilic insert is configured receive and evenly distribute the fluid through the distal tip by wicking the saline against gravity. The distal tip may generally include a plurality of ports or apertures configured to allow the fluid to pass therethrough, or weep, from the interior chamber to an external surface of the distal tip. The inflatable balloon, upon inflation, is shaped and sized so as to maintain the hydrophilic insert in contact with the interior surface of the distal tip wall, and specifically in contact with the one or more ports, such that the hydrophilic insert provides uniformity of saline distribution to the ports. Accordingly, upon positioning the distal tip within a target site (e.g., tissue cavity to be ablated), the inner balloon can be inflated to transition the distal tip to the expanded configuration, and the electrode array can be activated. Fluid can then be delivered to the interior chamber, specifically collecting in the hydrophilic insert, and the fluid weeping through the ports to the outer surface of the distal portion is able to carry energy from electrode array, thereby creating a virtual electrode. Accordingly, upon the fluid weeping through the ports, a pool or thin film of fluid is formed on the exterior surface of the distal tip and is configured to ablate surrounding tissue via the RF energy carried from the electrode array.

It should be noted the devices of the present disclosure are not limited to such post-surgical treatments and, as used herein, the phrase “body cavity” may include non-surgically created cavities, such as natural body cavities and passages, such as the ureter (e.g. for prostate treatment), the uterus (e.g. for uterine ablation or fibroid treatment), fallopian tubes (e.g. for sterilization), and the like. Additionally, or alternatively, tissue ablation devices of the present disclosure may be used for the ablation of marginal tissue in various parts of the body and organs (e.g., lungs, liver, pancreas, etc.) and is not limited to treatment of breast cancer.

It should be further noted that the device of the present disclosure can further be used during a surgical procedure, such as preparation for an orthopedic implant, in which the device is configured to selectively coagulate one or more pockets prepared within bone tissue for holding an implant so as to prevent or stop fluid accumulation (e.g., blood from vessel(s)) as a result of the implant preparation.

### BRIEF DESCRIPTION OF THE DRAWINGS

Features and advantages of the claimed subject matter will be apparent from the following detailed description of embodiments consistent therewith, which description should be considered with reference to the accompanying drawings, wherein:

FIGS. 1A and 1B are schematic illustrations of an ablation system consistent with the present disclosure;

FIG. 2 is a perspective view of one embodiment of an ablation device compatible with the system of FIG. 1A;

FIG. 3 is an enlarged view of the expandable distal tip assembly of the device of FIG. 2 in greater detail;

FIG. 4 is sectional view of the expandable distal tip assembly illustrating the nonconductive tip and the electrode array; and

FIGS. 5A and 5B are sectional views of the tip illustrating transitioning of the distal tip from a collapsed configuration (FIG. 5A) to an expanded configuration (FIG. 5B).

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

Tumors, both benign and malignant, are commonly treated and destroyed via surgical intervention, as surgery often offers the greatest chance for complete removal and cure, especially if the cancer has not metastasized. However, after the tumor is destroyed, a hollow cavity may remain, wherein tissue surrounding this cavity and surrounding the original tumor site can still leave abnormal or potentially cancerous cells that the surgeon fails, or is unable, to excise. This surrounding tissue is commonly referred to as “margin tissue” or “marginal tissue”, and is the location within a patient where a reoccurrence of the tumor may most likely occur.

Some alternative treatments to using radiation therapy include the use of ablation devices to be inserted within cavitary excisional beds and deliver radiofrequency (RF) energy to marginal tissue surrounding the cavity following the procedure. For example, one type of proposed ablation applicator includes a long rigid needle-based electrode applicator for delivery of RF energy to marginal tissue upon manual manipulation by a surgeon or operator. Another type of ablation application includes an umbrella-type array of electrodes jointly connected to one another and deployable in an umbrella-like fashion to deliver RF energy.

While current ablation devices may provide some form tissue ablation, none have proven to meet all needs and circumstances encountered when performing marginal cavity tissue ablation. For example, in certain instances, it may be desirable to create a non-uniform ablation within a tissue cavity. In some instances, vital organs or critical internal/external structures (e.g., bone, muscle, skin, etc.) may be in close proximity to a tissue cavity and any unintended exposure to RF energy could have a negative impact. Current RF ablation devices are unable to provide precise control over the emission of RF energy such that they lack the ability to effectively prevent emission from reaching vital organs or important internal/external structures during the ablation procedure. In particular, the long rigid needle-based electrode RF applicators generally require the surgeon or operator to manually adjust needle locations, and possibly readjust several electrodes multiple times, in order to control an ablation, which may lead to inaccuracy and difficulty in directing RF emission. The umbrella array RF applicators are limited by their physical geometry, in that the umbrella array may not be designed to fit within a cavity. Additionally, or alternatively, the uniform potential distribution of an umbrella array, as a result of the electrodes being jointly connected to one another, results in a tissue ablation geometry that is not adjustable without physically moving the umbrella array, thus resulting in similar problems as long rigid needle-based RF applicators.

By way of overview, the present disclosure is generally directed to a tissue ablation system including an ablation device having an expandable applicator tip configured to emit radio frequency (RF) energy for ablation and destruction of a target tissue. The tissue ablation system of the present disclosure can be used during an ablation procedure to destroy the thin rim of marginal tissue around the cavity in a targeted manner. In particular, the present disclosure is generally directed to a cavitary tissue ablation system including an ablation device to be delivered into a tissue cavity and configured to emit non-ionizing radiation, such as radiofrequency (RF) energy, in a desired shape or pattern so as to deliver treatment for the ablation and destruction of a targeted portion of marginal tissue around the tissue cavity. The ablation device includes an expandable applicator tip configured to emit the RF energy in a desired pattern. The applicator tip includes a non-conductive flexible material configured to transition between a collapsed configuration, in which the tip can be delivered to a target site (i.e., a cavity or pocket), and an expanded configuration, in which the tip surface can better conform to the contour of the target tissue to be ablated, thereby allowing for improved contact and ablation/coagulation performance of the ablation device.

The system of the present invention is configured to provide a user with custom ablation shaping, which includes the creation of custom, user-defined ablation geometries depending on the target site. In particular, rather than simply providing a universal RF ablation shape or profile, the system allows for a user to customize the emission of energy to a targeted portion of marginal tissue within the cavity, which is particularly useful in instances in which non-uniform ablation is desired. The customized emission of energy may include a specific shape or geometry of emission, as well as time and depth of penetration of RF energy.

The devices, systems, and methods of the present disclosure can help to ensure that all microscopic disease in the local environment has been treated. This is especially true in the treatment of tumors that have a tendency to recur. Furthermore, by providing custom ablating shaping, in which the single ablation device may provide numerous RF energy emission shapes or profiles, the system of the present invention allows for non-uniform ablation to occur. This is particularly useful in controlling ablation shape so as to avoid vital organs and any critical internal/external structures (e.g., bone, muscle, skin) in close proximity to the tumor site, while ensuring that residual marginal tissue within the local environment has been treated.

The tissue ablation device of the present invention generally includes a probe including an elongated shaft configured as a handle and adapted for manual manipulation and a flexible nonconductive distal portion or tip coupled to the shaft. The nonconductive distal tip is formed from a material having a low durometer and is deformable, thereby allowing for the distal tip to transition between a collapsed configuration, in which the distal tip has a first diameter, and an expanded configuration, in which the distal tip has a second diameter greater than the first diameter. The distal tip generally includes an interior chamber in which an inflatable inner balloon is positioned. The configuration of the distal tip is generally dependent on the current state of the inner balloon. In other words, the distal tip transitions to the expanded configuration in response to inflation of the inner balloon. Similarly, the distal tip transitions to the collapsed configuration in response to deflation of the inner balloon.

The distal tip includes an electrode array positioned along an external surface thereof. The distal tip, including the electrode array, can be delivered to and maneuvered within a tissue cavity (e.g., formed from tumor removal) when the distal tip is in the collapsed configuration and, upon transition to the expanded configuration, the distal tip is configured to ablate marginal tissue (via RF energy) immediately surrounding the tissue cavity in order to minimize recurrence of the tumor. The ablation device of the present invention is further configured to provide a user with custom ablation shaping, which includes the creation of custom, user-defined ablation geometries or profiles.

In one aspect, the electrode array includes a plurality of conductive wires electrically isolated and independent from one another. This design allows for each conductive wire to receive energy in the form of electrical current from a source (e.g., RF generator) and emit RF energy in response. The system may include a device controller, for example, configured to selectively control the supply of electrical current to each of the conductive wires. By allowing for independent control of each wire, the ablation system provides for custom ablation shaping to occur. In particular, the device controller allows for individual conductive wires, or a designated combination of conductive wires, to be controlled so as to result in the activation (e.g., emission of RF energy) of corresponding portions of the electrode array.

The device controller can selectively activate one or more of the electrode array portions (e.g., control the supply of electrical current to specific sets of conductive wires) so as to provide targeted delivery of RF energy from the ablation device in a desired pattern or shape. In addition to customizing the shape or geometry of RF energy emission from the ablation device, the device controller may be further configured to control particular ablation parameters, such as control of timing of the emission (e.g., length of time, intervals, etc.) as well as the depth of RF energy penetration.

In some embodiments, the ablation device is configured to provide RF ablation via a virtual electrode arrangement, which includes distribution of a fluid along an exterior surface of the distal tip and, upon activation of the electrode array, the fluid may carry, or otherwise promote, energy emitted from the electrode array to the surrounding tissue. For example, the nonconductive distal tip of the ablation device includes the interior chamber retaining at least the inner balloon, which may essentially act as a spacing member, and a hydrophilic insert surrounding a inner balloon. The interior chamber of the distal tip is configured to receive and retain a fluid (e.g., saline) therein from a fluid source. The hydrophilic insert is configured receive and evenly distribute the fluid through the distal tip by wicking the saline against gravity. The distal tip may generally include a plurality of ports or apertures configured to allow the fluid to pass therethrough, or weep, from the interior chamber to an external surface of the distal tip. The inflatable balloon, upon inflation, is shaped and sized so as to maintain the hydrophilic insert in contact with the interior surface of the distal tip wall, and specifically in contact with the one or more ports, such that the hydrophilic insert provides uniformity of saline distribution to the ports. Accordingly, upon positioning the distal tip within a target site (e.g., tissue cavity to be ablated), the inner balloon can be inflated to transition the distal tip to the expanded configuration, and the electrode array can be activated. Fluid can then be delivered to the interior chamber, specifically collecting in the hydrophilic insert, and the fluid weeping through the ports to the outer surface of the distal portion is able to carry energy from electrode array, thereby creating a virtual electrode. Accordingly, upon the fluid weeping through the ports, a pool or thin film of fluid is formed on the exterior surface of the distal tip and is configured to ablate surrounding tissue via the RF energy carried from the electrode array.

It should be noted the devices of the present disclosure are not limited to such post-surgical treatments and, as used herein, the phrase “body cavity” may include non-surgically created cavities, such as natural body cavities and passages, such as the ureter (e.g. for prostate treatment), the uterus (e.g. for uterine ablation or fibroid treatment), fallopian tubes (e.g. for sterilization), and the like. Additionally, or alternatively, tissue ablation devices of the present disclosure may be used for the ablation of marginal tissue in various parts of the body and organs (e.g., lungs, liver, pancreas, etc.) and is not limited to treatment of breast cancer.

It should be further noted that the device of the present disclosure can further be used during a surgical procedure, such as preparation for an orthopedic implant, in which the device is configured to selectively coagulate one or more pockets prepared within bone tissue for holding an implant so as to prevent or stop fluid accumulation (e.g., blood from vessel(s)) as a result of the implant preparation.

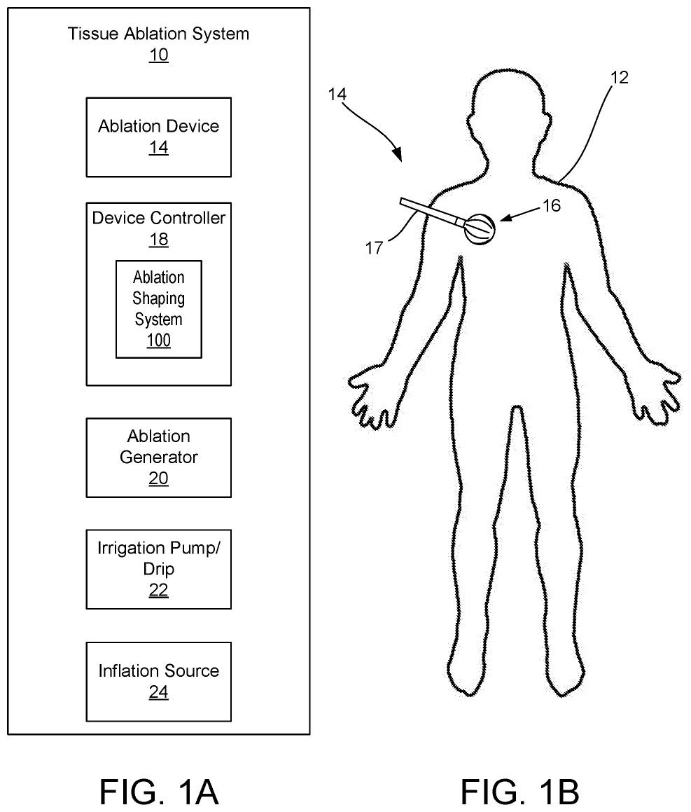

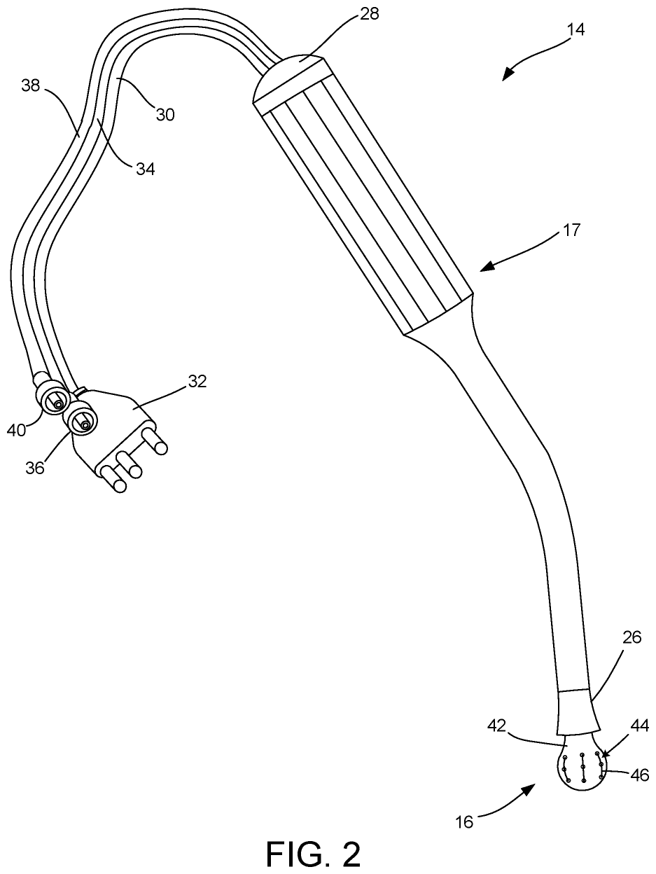

FIGS. 1A and 1B are schematic illustrations of an ablation system 10 for providing targeted ablation of marginal tissue during a tumor removal procedure in a patient 12. The ablation system 10 generally includes an ablation device 14, which includes a probe having a distal tip or portion 16 and an elongated catheter shaft 17 to which the distal tip 16 is connected. The catheter shaft 17 may generally include a nonconductive elongated member including a fluid delivery lumen, in addition to other lumens as described in greater detail herein. The ablation device 14 may further be coupled to a device controller 18 and an ablation generator 20 over an electrical connection (electrical line 30 shown in FIG. 2), and an irrigation pump or drip 22 over a fluid connection (fluid line 34 shown in FIG. 2), and an inflation source 24 over a connection (connection line 38 shown in FIG. 2).

The device controller 18 may include hardware/software configured to provide a user with the ability to control electrical output to the electrosurgical device 14 in a manner so as to control ablation output to a wound site for treating chronic wound tissue. For example, the ablation device may be configured to operate at least in a “bipolar mode” based on input from a user (e.g., surgeon, clinician, etc.) resulting in the emission of radiofrequency (RF) energy in a bipolar configuration. In some embodiments, the device 14 may be configured to operate in other modes, such as a “measurement mode”, in which data can be collected, such as certain measurements (e.g., temperature, conductivity (impedance), etc.) that can be taken and further used by the controller 18 so as to provide an estimation of the state of tissue during a wound treatment procedure. Further still, the device controller 18 may include a custom ablation shaping (CAS) system 100 configured to provide a user with custom ablation shaping, which includes the creation of custom, user-defined ablation geometries or profiles from the device 14. The CAS system 100 may further be configured to provide ablation status mapping and ablation shaping based on real-time data collection (e.g., measurements) collected by the device.

The features and functions of the controller 18 and CAS system 100 are described in U.S. application Ser. No. 15/419,256, filed Jan. 30, 2017 (Publication No. 2017/0215951), U.S. application Ser. No. 15/419,269, filed Jan. 30, 2017 (Publication No. 2017/0215947), and application Ser. No. 15/902,398, filed Feb. 22, 2017, the contents of each of which are incorporated by reference herein in their entireties.

FIG. 2 is a perspective view of one embodiment of an ablation device 14. As previously described, the electrosurgical device 14 includes a probe 17 including an elongated shaft configured as a handle and adapted for manual manipulation. Accordingly, as shown in FIG. 2, the probe 17 is in the form of a handle having a distal end 26 to which the tip assembly 16 is coupled and a proximal end 28. As shown, the proximal end 28 of the probe 17 may be coupled to the generator 20, the irrigation pump 22, and the inflation source 24 via connection lines or fittings. For example, the probe 17 is coupled to the generator 20 via an electrical line 30, coupled to the irrigation pump 22 via a fluid line 34, and coupled to the inflation source 24 via a connection line 38. Each of the electrical line 30, fluid line 34, and connection line 38 may include an adaptor end 32, 36, 40 configured to couple the associated lines with a respective interface on the generator 20, irrigation pump 22, and inflation source 24.

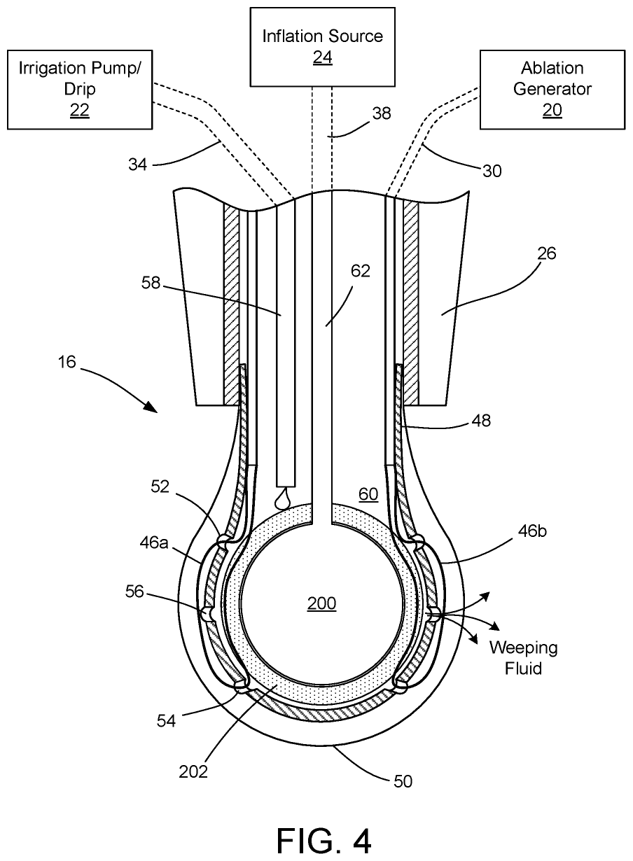

In some examples, the electrosurgical device 14 may further include a user interface (not shown) serving as the device controller 18 and in electrical communication with at least one of the generator 20, the irrigation pump 22, and/or inflation source 24, and the electrosurgical device 14. The user interface 28 may include, for example, selectable buttons for providing an operator with one or more operating modes with respect to controlling the energy emission output of the device 14, as will be described in greater detail herein. For example, selectable buttons may allow a user to control electrical output to the electrosurgical device 14 in a manner so as to control the ablation of a target tissue. Furthermore, in some embodiments, selectable buttons may provide an operator to control the delivery of fluid from the irrigation pump 22 and/or activation of the inflation source 24 to control inflation of an inner balloon within the distal tip 16 (shown in FIG. 4).

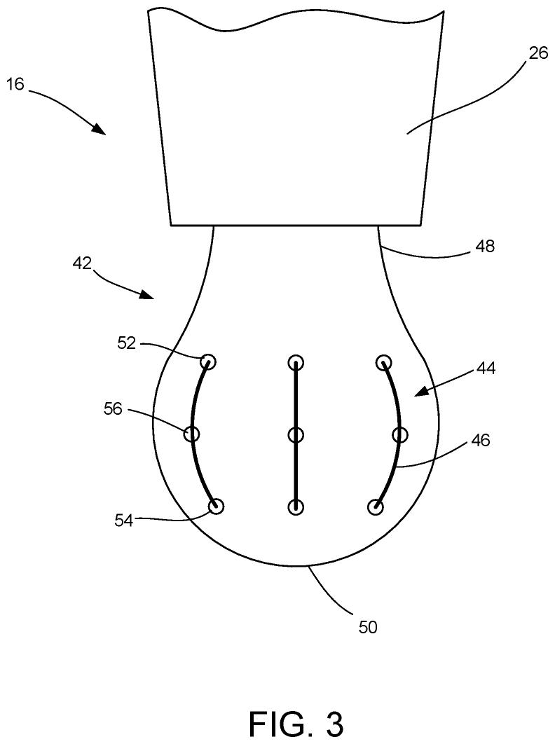

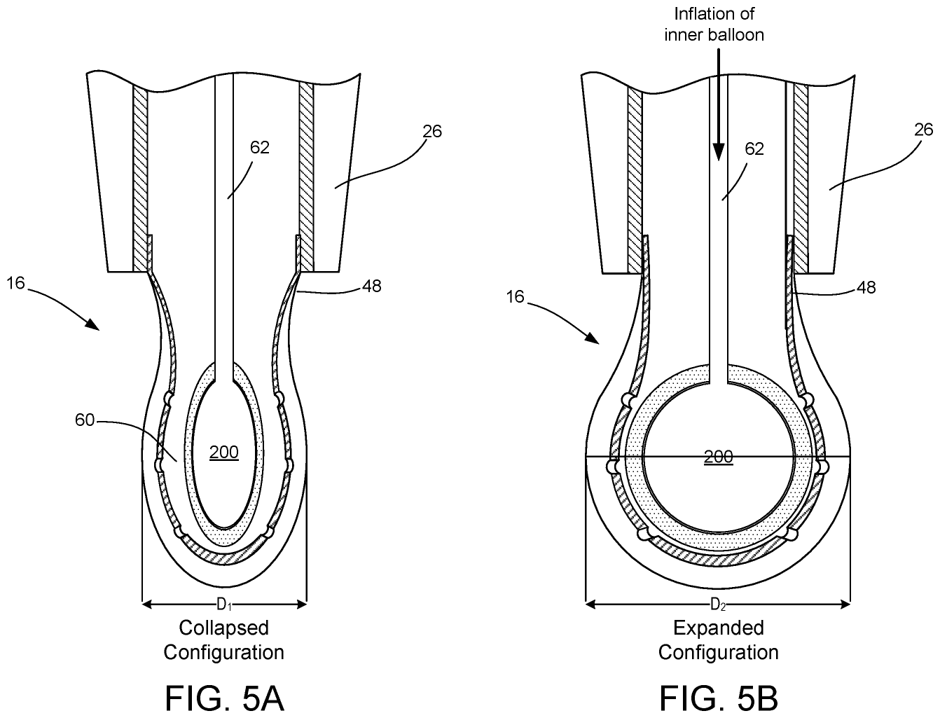

The tip assembly 16 includes a nonconductive tip 42 extending from the distal end 26 of the probe shaft 17 and an electrode array 44 comprising a plurality of independent conductive wires 46 extending along an external surface of the nonconductive tip 42. As will be described in greater detail herein, the tip assembly 16 is flexible and generally formed from a low durometer material. More specifically, the nonconductive tip 42 and electrode array are generally flexible and configured to transition from a collapsed configuration (shown in FIG. 5A), in which the tip 42 has a smaller diameter and can be more easily delivered to a target site, to an expanded configuration (e.g., generally spherical as shown in FIGS. 3, 4, and 5B) upon a inflation of an inner balloon member. The expansion of the tip assembly 16 allows for the tip assembly to conform to the contour of a target tissue, allowing for improved contact and ablation/coagulation performance.

FIG. 3 is an enlarged view of the expandable tip assembly 16 and FIG. 4 is sectional view of the expandable tip assembly 16 illustrating the nonconductive tip and the electrode array relative to one another. As shown, the nonconductive tip 42 includes a proximal end 48 coupled to the distal end 26 of the probe shaft 17 and a distal end 50. As will be described in greater detail herein, the nonconductive tip 42 includes a flexible body configured to transition from a collapsed configuration (shown in FIG. 5A) to an expanded configuration (shown in FIG. 5B) upon inflation of an inner balloon member. Upon deflation of the inner balloon member, the nonconductive tip 42 is configured to transition back to the collapsed configuration. Accordingly, the nonconductive tip 42 may include an elastomeric or shape memory material. As shown in FIGS. 3 and 4, the nonconductive tip 42 has a generally spherical shape when in the expanded configuration. Upon application of a force (e.g., pressing of the tip 42 against a wound bed or the like), the nonconductive tip 42 is configured to flex and transition into a deformed state, where portions of the nonconductive tip 42 can become deformed such that nonconductive tip assumes a compressed shape. It should be noted that the ablation device 14 and tip assembly 16 may be similarly configured as the ablation device and tip assembly described in U.S. application Ser. No. 15/646,697, filed Jul. 11, 2017 (Publication No. 2018/0014880), the content of which is incorporated by reference herein in its entirety.

As shown in FIGS. 3 and 4, the nonconductive tip 42 includes plurality of proximal ports 52 and distal ports 54 in communication with the at least one lumen of the probe shaft 17. The proximal ports 52 and distal ports 54 generally serve as openings through which conductive wires 46 of the electrode array 44 may pass. For example, each of the plurality of wires 46 passes through an associated one of the proximal ports and through a corresponding one of the proximal ports. Accordingly, the number of proximal ports 52 and distal ports 54 may generally be equal to the number of conductive wires 46, such that each conductive wire 46 can extend through a different distal port 54, which allows the conductive wires 46 to remain electrically isolated from one another. In other examples, one or more conductive wires can extend through the same distal port 54. The nonconductive tip 42 may further include one or more ports 56 configured to allow passage of fluid from the within the nonconductive tip 42 to an external surface of the nonconductive tip 42, as will be described in greater detail herein.

Upon passing through a distal port 54, each conductive wire 46 can extend along an external surface of the nonconductive tip 42. In some examples, the length of the conductive wire 46 extending along the external surface is at least 20% (e.g., at least, 50%, 60%, 75%, 85%, 90%, or 99%) of the length of the nonconductive tip 42. The conductive wire 46 can then re-enter the nonconductive tip 42 through a corresponding proximal port 52. For example, as shown in FIG. 4, conductive wire 46a passes through distal port 54, extends along a length of the external surface of the nonconductive tip 42, and passes through an associated proximal port 52 and into a cavity of the nonconductive tip 42, while conductive wire 46b is electrically isolated from conductive wire 46a in that it passes through its own associated proximal and distal ports. The wires 46 are configured to receive energy in the form of electrical current from the RF generator 20 and emit RF energy in response. The conductive wires 46 can be formed of any suitable conductive material (e.g., a metal such as stainless steel, nitinol, or aluminum).

As shown, one or more of the conductive wires 46 can be electrically isolated from one or more of the remaining conductive wires, such that the electrical isolation enables various operation modes for the electrosurgical device 14. For example, electrical current may be supplied to one or more conductive wires in a bipolar mode, a unipolar mode, or a combination bipolar and unipolar mode. In the unipolar mode, ablation energy is delivered between one or more conductive wires of the electrode array 44 and a return electrode 15, for example. In bipolar mode, energy is delivered between at least two of the conductive wires, while at least one conductive wire remains neutral. In other words, at least, one conductive wire functions as a grounded conductive wire (e.g., electrode) by not delivering energy over at least one conductive wire.

Since each conductive wire 46 in the electrode array 44 is electrically independent, each conductive wire 46 can be connected in a fashion that allows for impedance measurements using bipolar impedance measurement circuits. For example, the conductive wires can be configured in such a fashion that tetrapolar or guarded tetrapolar electrode configurations can be used. For instance, one pair of conductive wires could function as the current driver and the current return, while another pair of conductive wires could function as a voltage measurement pair. Accordingly, a dispersive ground pad can function as current return and voltage references. Their placement dictate the current paths and thus having multiple references can also benefit by providing additional paths for determining the ablation status of the tissue.

As previously described, the ablation device 14 is configured to provide RF ablation via a virtual electrode arrangement. In particular, energy conducted by one or more of the wires 46 is carried by the fluid weeping from the nonconductive tip 42, thereby creating a virtual electrode. For example, the nonconductive tip 42 includes an interior chamber 60 retaining at least an inner balloon member 200 therein, which may essentially act as a spacing member, and a hydrophilic insert 202 surrounding a inner balloon member 200. As shown, the probe shaft 17 includes a fluid lumen 58 coupled to the irrigation pump or drip 22 via the fluid line 34 and is configured to receive conductive fluid therefrom. The hydrophilic insert 202 is configured receive and evenly distribute the conductive fluid from the fluid lumen 58 within the interior chamber 60 by wicking the saline against gravity. The saline within the chamber 60 may be distributed from the hydrophilic insert 202 to an external surface of the tip 42 through the one or more ports 56 and/or the ports (e.g., to the proximal ports 52 and distal ports 54). The saline weeping through the ports 56 and/or ports 52, 54 to an outer surface of the nonconductive tip 42 is able to carry electrical current from the electrode array 44, such that energy is transmitted from the electrode array 44 to a target tissue by way of the saline, thereby creating a virtual electrode. The specific arrangement and features of components of the ablation device, including conductive wires, inner balloon member (i.e., spacing member), and hydrophilic insert, are described in U.S. Pat. No. 9,848,936, the content of which is incorporated by reference herein in its entirety.

The probe shaft 17 further includes an inflation lumen 62 configured to be coupled to the inflation source 24 via the connection line 38. Accordingly, the inflatable balloon member 200 is in fluid communication with the inflation source 24 via the inflation lumen 62, such that, when the inflation source is activated, the inner balloon member 200 inflates. Upon inflation, the inner balloon member 200 is shaped and sized so as to maintain the hydrophilic insert 202 in contact with the interior surface of the distal tip wall, and specifically in contact with the one or more ports, such that the hydrophilic insert provides uniformity of saline distribution to the ports 56 and/or ports 52, 54. Accordingly, upon positioning the distal tip within a target site (e.g., tissue cavity to be ablated), the inner balloon member can be inflated to transition the distal tip to the expanded configuration, and the electrode array can be activated. Fluid can then be delivered to the interior chamber, specifically collecting in the hydrophilic insert, and the fluid weeping through the ports to the outer surface of the distal portion is able to carry energy from electrode array, thereby creating a virtual electrode. Accordingly, upon the fluid weeping through the ports, a pool or thin film of fluid is formed on the exterior surface of the distal tip and is configured to ablate surrounding tissue via the RF energy carried from the electrode array.

FIGS. 5A and 5B are sectional views of the distal tip illustrating transitioning of the distal tip from a collapsed configuration (FIG. 5A) to an expanded configuration (FIG. 5B). As shown, when in the collapsed configuration, the distal tip has a first diameter D₁ and, when in the expanded configuration, the distal tip has a second diameter D₂ that is greater than the first diameter D₁.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

What is claimed is:

1. A device comprising:
an expandable distal portion defining an interior chamber and also defining a plurality of ports;
an inflatable member disposed within the interior chamber of the distal portion, the inflatable member configured to transition from a collapsed configuration to an expanded configuration and cause the distal portion to correspondingly transition from a collapsed configuration to an expanded spherical shape;
a hydrophilic member disposed within the interior chamber between an exterior surface of the inflatable member and an interior surface of the distal portion, the hydrophilic member configured to receive and distribute a conductive fluid to the plurality of ports; and
a conductive wire disposed along at least a portion of an exterior surface of the distal portion.

2. The device of claim 1, further comprising handle including a lumen for receiving the conductive fluid, wherein the lumen is in fluid communication with the interior chamber of the distal portion.

3. The device of claim 1, wherein one or more of the plurality of ports is configured to allow passage of the conductive fluid to an exterior surface of the distal portion.

4. The device of claim 3, wherein, upon receipt of an electric current, the conductive wire is configured to conduct radiofrequency (RF) energy to be carried by the conductive fluid passing through one or more of the plurality of ports for ablation of a tissue.

5. The device of claim 3, wherein the plurality of ports comprises one or more medial ports for allowing passage of the conductive fluid.

6. The device of claim 1, wherein the conductive wire is substantially aligned with at least one of the plurality of ports.

7. The device of claim 1, wherein the plurality of ports comprises a plurality of proximal ports and distal ports, wherein the conductive wire passes through at least one of the proximal ports and through a corresponding one of the distal ports such that a portion of the conductive wire has a length that extends along the exterior surface of the distal portion between the corresponding proximal and distal ports.

8. The device of claim 7, wherein the conductive wire is one of a plurality of conductive wires, each of the plurality of conductive wires is disposed along at least a portion of the exterior surface of the distal portion.

9. The device of claim 8, wherein each of the plurality of conductive wires passes through at least one of the proximal ports and through a corresponding one of the distal ports, wherein each of the plurality of proximal ports corresponds to a separate one of the plurality of distal ports such that a portion of a conductive wire passing through a set of corresponding proximal and distal ports has a length that extends along the exterior surface of the distal portion between the corresponding proximal and distal ports.

10. The device of claim 1, wherein the distal portion comprises a nonconductive material.

11. A device comprising:
an expandable distal portion defining an interior chamber and also defining a plurality of ports, the expandable distal portion configured to transition from a collapsed configuration to an expanded spherical shape;
a hydrophilic member disposed within the interior chamber of the distal portion, the hydrophilic member configured to receive and distribute a conductive fluid to the plurality of ports; and
a conductive wire disposed along at least a portion of an exterior surface of the distal portion and configured to conduct energy to be carried by a conductive fluid passing through one or more of the plurality of ports.

12. The device of claim 11, further comprising an inflatable balloon disposed within the interior chamber of the distal portion, the inflatable balloon configured to transition from a collapsed configuration to an expanded configuration and cause the distal portion to correspondingly transition from the collapsed configuration to the expanded spherical shape.

13. The device of claim 11, further comprising handle including a lumen for receiving the conductive fluid, wherein the lumen is in fluid communication with the interior chamber of the distal portion.

14. The device of claim 11, wherein one or more of the plurality of ports is configured to allow passage of the conductive fluid to an exterior surface of the distal portion.

15. The device of claim 14, wherein, upon receipt of an electric current, the conductive wire is configured to conduct radiofrequency (RF) energy to be carried by the conductive fluid passing through one or more of the plurality of ports for ablation of a tissue.

16. The device of claim 14, wherein the plurality of ports comprises a plurality of proximal ports, medial ports, and distal ports.

17. The device of claim 16, wherein the conductive wire passes through at least one of the proximal ports and through a corresponding one of the distal ports such that a portion of the conductive wire has a length that extends along the exterior surface of the distal portion between the corresponding proximal and distal ports and is substantially aligned with a corresponding medial port.

18. The device of claim 17, wherein the conductive wire is one of a plurality of conductive wires, each of the plurality of conductive wires is disposed along at least a portion of the exterior surface of the distal portion.

19. The device of claim 18, wherein each of the plurality of conductive wires passes through at least one of the proximal ports and through a corresponding one of the distal ports, wherein each of the plurality of proximal ports corresponds to a separate one of the plurality of distal ports such that a portion of a conductive wire passing through a set of corresponding proximal and distal ports has a length that extends along the exterior surface of the distal portion between the corresponding proximal and distal ports and is substantially aligned with a corresponding medial port.

20. The device of claim 11, wherein the distal portion comprises a nonconductive material.
